(19) United States
(12) Patent Application Publication
(10) Pub. No.: US 2005/0096534 A1
(43) Pub. Date: May 5, 2005

(54) Systems and methods for calibrating coil sensitivity profiles

(76) Inventors: Yudong Zhu, Clifton Park, NY (US); Charles Lucian Dumoulin, Ballston Lake, NY (US)

Correspondence Address: GENERAL ELECTRIC COMPANY (PCPI);C/O FLETCHER YODER, P. O. BOX 692289, HOUSTON, TX, 77269-2289, US

(21) Appl. No.: 10/699,059

(22) Filed: Oct. 31, 2003

(51) Int. Cl. A61B 5/05; G01V 3/00

(52) U.S. Cl. 600/422; 324/318

(57) ABSTRACT

A method for calibrating coil sensitivity profiles is described. The method includes generating reference sensitivity maps for each coil, imaging a subject, interleaving, with the imaging of the subject, imaging of at least one fiducial mark provided with each coil, and deriving, based on the coil positioning and coil loading, actual sensitivity maps from the reference sensitivity maps.

### BACKGROUND OF THE INVENTION

This invention relates generally to magnetic resonance imaging (MRI) systems and more particularly, to systems and methods for calibrating coil sensitivity maps or profiles of coils used within MRI systems.

MRI is a technique that is capable of providing three-dimensional imaging of an object, such as the heart or brain, of a patient. At least some known MRI systems include a main or primary magnet that provides a polarizing magnetic field Bₒ, and include gradient coils and radio frequency (RF) coils, which are used for spatial encoding, exciting and detecting nuclei of the patient during imaging. Typically, the main magnet provides a homogeneous magnetic field in an internal region within the main magnet, for example, within an air space defined within a solenoid, or within an air gap defined between magnetic pole faces of a C-type magnet. The patient or an object to be imaged is positioned in the homogeneous field region such that the gradient coils and the RF coils are typically located external to the patient or object, while being inside the geometry of the main magnet surrounding the air space.

In MRI, the uniform magnetic field Bₒ is applied to the object along a Z-axis of a Cartesian coordinate system, the origin of which is within the object. The uniform magnetic field Bₒ facilitates aligning nuclear spins of nuclei of the object. In response to RF pulses of a resonant frequency, that are orientated within an X-Y plane of the Cartesian coordinate system, the nuclei resonate at their Larmor frequencies. During an imaging sequence, an RF pulse centered about the Larmor frequency and having a selected bandwidth is applied to the object at substantially the same time a magnetic field gradient G_{z} is applied along the Z-axis. Gradient field G_{z} subjects nuclei in a slice having a limited width through the object to the resonant frequency and thus the nuclei are excited into resonance.

After excitation of the nuclei in the slice, magnetic field gradients Gₓ and G_{y} are applied along an X-axis and Y-axis, respectively, of the Cartesian coordinate system. The gradient Gₓ along the X-axis causes the nuclei to precess at different frequencies depending on their position along the X-axis, that is, Gₓ spatially encodes the precessing nuclei by frequency, a process referred to as frequency encoding. The Y-axis gradient G_{y} is incremented through a series of values and encodes the nuclei along the Y-axis into the rate of change of phase of the precessing nuclei as a function of gradient amplitude, a process referred to as phase encoding.

Two known methods, Simultaneous Acquisition of Spatial Harmonics (SMASH) imaging in a time domain or k-space, and Sensitivity Encoded (SENSE) imaging in a spatial domain, change sequential data acquisition of the MRI system into a partially parallel process by using a phased array, thereby reducing scan time as compared to methods using a sequential data acquisition technique. Within these two methods, data sampled below a Nyquist sampling rate may be recovered if the sensitivity profiles of the RF coils can provide enough spatial information to either interpolate the data in the time domain or unwrap the data in the spatial domain.

The SMASH method recognizes the equivalence between phase encoding with the gradient G_{y} and composite coil sensitivity profiles inherent in the RF coils, and uses a numerical fitting routine to interpolate a decimated number of phase encoding steps and thus, reducing scan times. Initially, coil sensitivity profiles of each of the RF coils are derived from a separate data acquisition performed by using the MRI system. Second, by using numerical fitting and computation, such as minimum least square or gradient-descent algorithms, coefficients or weights of linear combinations that compose the desired or optimal coil sensitivity profiles from the RF coils are numerically derived. Third, using composite harmonics to interpolate decimated phase encoding steps, the data is sampled at the Nyquist frequency. Fourth, a Fast Fourier Transform (FFT) of the composite harmonics provides a non-aliasing MR image. The SENSE method also uses precise coil sensitivity profiles of all the RF coils.

Methods used to obtain the coil sensitivity profiles of the RF coils involve additional calibration imaging steps that produce low-resolution images of coil sensitivity profiles. However, the calibration imaging steps may incur significant calibration time overhead and the diagnostic imaging quality may suffer because images produced by the calibration steps a) may not provide coil sensitivity information at signal voids where there are no spins, or b) may lack adequate update to capture profile alterations due to coil orientation and/or coil loading changes between calibration imaging and diagnostic imaging. Issues in a) and b) pose challenges in such applications as cardiac imaging where signal voids present in surrounding areas of a beating heart and coil orientation and/or coil loading may alter due to motion either of the object or the patient.

### BRIEF DESCRIPTION OF THE INVENTION

In one aspect, a method for calibrating coil sensitivity profiles is provided. The method includes generating reference sensitivity maps for each coil, imaging a subject, interleaving, with the imaging of the subject, imaging of at least one fiducial mark provided with each coil, and deriving, based on the coil positioning and coil loading, actual sensitivity maps from the reference sensitivity maps.

In another aspect, a magnetic resonance imaging system is provided. The magnetic resonance imaging system includes a coil array configured to receive a plurality of signals to generate magnetic resonance images, where the coil array is configured to obtain partial gradient phase encoding signals from a subject, to intermittently receive signals from at least one fiducial mark provided with each coil of the coil array, and to intermittently receive signals while obtaining the partial gradient phase encoding signals. The magnetic resonance imaging system also includes an image reconstructor configured to update sensitivity maps by using the intermittently received signals and reference sensitivity maps, where the image reconstructor is further configured to construct magnetic resonance images based on the updated sensitivity maps and the partial gradient phase encoding signals.

In yet another aspect, a magnetic resonance imaging system is provided. The magnetic resonance imaging system includes a coil array configured to receive a plurality of signals, and a controller configured to generate sensitivity maps from the plurality of signals. The coil array is further configured to collect partial gradient phase encoding signals from a subject, to intermittently receive signals from at least one fiducial mark provided with each coil of the coil array, and to intermittently receive while obtaining the partial gradient phase encoding signals.

### BRIEF DESCRIPTION OF THE DRAWINGS

FIG. 1 illustrates an exemplary embodiment of a magnetic resonance imaging (MRI) system.

FIG. 2 illustrates an embodiment of coil arrays that are arranged to detect MR signals from a subject placed within the MRI system of FIG. 1.

FIG. 3 illustrates a flowchart of an embodiment of a method for calibrating coil sensitivity profiles that is implemented by using the MRI system of FIG. 1.

FIG. 4 illustrates a front view and a side view of an embodiment of a surface of a coil of the coil arrays of FIG. 2.

### DETAILED DESCRIPTION OF THE INVENTION

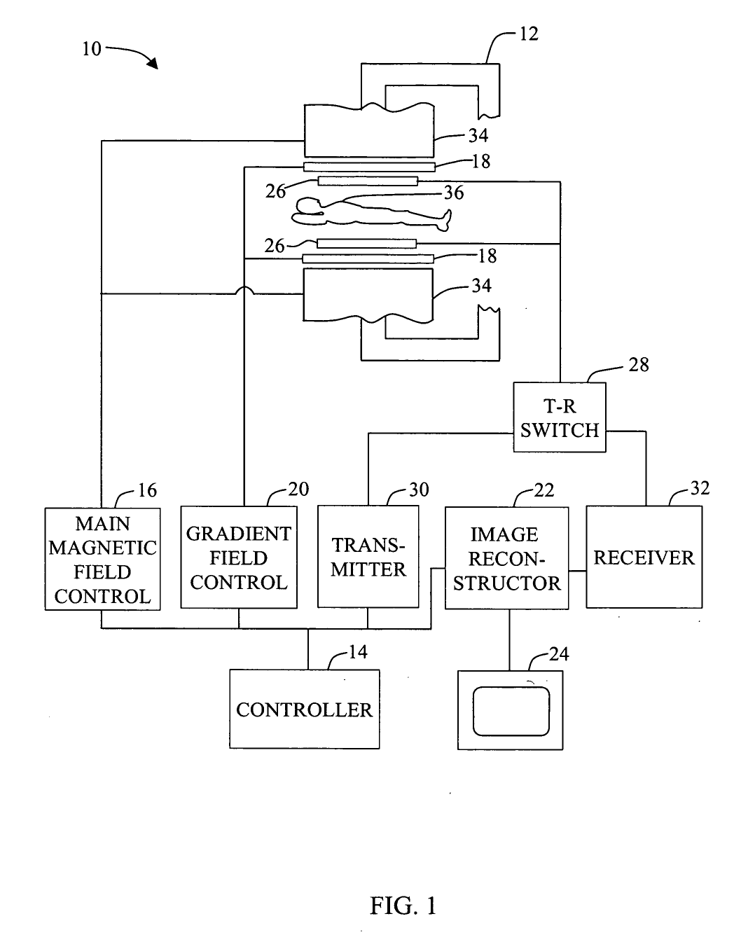

FIG. 1 illustrates an embodiment of a magnetic resonance imaging (MRI) system 10 in which systems and methods for calibrating coil sensitivity profiles are implemented. MRI system 10 includes an electromagnet 12, a controller 14, a main magnetic field control 16, a gradient coil sub-system 18, a gradient field control 20, an image reconstructor 22, a display device 24, coil arrays 26, a T-R (transmit-receive) switch 28, a transmitter 30, and a receiver 32.

The term controller, as used herein, is not limited to just those integrated circuits referred to in the art as computers, but broadly refers to processors, microcontrollers, microcomputers, programmable logic controllers, application specific integrated circuits, and other programmable circuits, and these terms are used interchangeably herein. Although a C-type electromagnet 12 is illustrated, other shapes of electromagnets, such as an electromagnet that completely surrounds a subject 36, such as a patient or a phantom, can be used instead.

In one embodiment, electromagnet 12 produces a strong main magnetic field Bₒ across a gap between pole pieces 34 of the electromagnet. In use of MRI system 10, a subject 36 or alternatively an object, such as heart or lungs, to be imaged is placed in the gap between pole pieces 34 on a suitable support (not shown). The strength of the magnetic field Bₒ in the gap between pole pieces 34, and hence in subject 36, is controlled by controller 14 via main magnetic field control 16, which controls a supply of energizing current to electromagnet 12.

Gradient coil sub-system 18, having one or more gradient coils, is provided so a magnetic gradient can be imposed on the magnetic field Bₒ in the gap between poles pieces 34 in any one or more of three orthogonal directions x, y, and z. Gradient coil sub-system 18 is energized by gradient field control 20 that also is under the control of controller 14.

Each coil array 26 includes a plurality of coils arranged to simultaneously detect MR signals from subject 36. Coil arrays 26 are selectably interconnected to one of transmitter 30 or receiver 32 by T-R switch 28. Transmitter 30 and T-R switch 28 are under the control of controller 14 so that RF field pulses or signals are generated by transmitter 30 and selectively applied by coil array 26 to subject 36 for excitation of magnetic resonance in the subject. While these RF excitation pulses are being applied to subject 36, T-R switch 28 also is actuated so as to de-couple receiver 32 from coil array 26.

Following application of the RF pulses, T-R switch 28 is again actuated to de-couple coil array 26 from transmitter 30 and to couple the coil array to receiver 32. Coil array 26 in this arrangement detects or senses the MR signals resulting from excited nuclei in subject 36 and passes the MR signals onto the receiver 32. These detected MR signals are in turn passed onto image reconstructor 22. Image reconstructor 22, under the control of controller 14, processes the MR signals to produce signals representative of an image of subject 36. In one embodiment, the image is reconstructed by applying a Fourier transformation on a composite MR signal in the k-space. The composite MR signal is a combination of MR signals of each coil in coil array 26. In an alternative embodiment, the image is reconstructed by applying a Fourier transformation on an individual MR signal from a coil in coil array 26. In yet another alternative embodiment, the image can be reconstructed by backprojecting the composite MR signal or alternatively, backprojection the individual MR signal. The processed signals representative of the image are sent onto a display device 24, such as a cathode ray tube, to provide a visual display of the image.

In operation, the magnetic field Bₒ generated by the electromagnet 12 is applied to subject 36 by convention along a Z-axis of a Cartesian coordinate system, the origin of which is within the subject. The magnetic field Bₒ being applied has the effect of aligning nuclear spins, of nuclei of subject 2, along the Z-axis. In response to RF pulses of a proper resonant frequency being generated by transmitter 30, that are orientated within an X-Y plane of the Cartesian coordinate system, the nuclei resonate at their Larmor frequencies. In a typical imaging sequence, an RF pulse centered about the Larmor frequency is applied to subject 36 at the same time a magnetic field gradient G_{z} is being applied along the Z-axis by means of gradient control sub-system 18. The gradient G_{z} causes nuclei in a slice with a limited width through subject 36 along the X-Y plane, to have the resonant frequency and to be excited into resonance.

After excitation of the nuclei in the slice, magnetic field gradients Gₓ and G_{y} are applied along x and y axes, respectively, of the Cartesian coordinate system. The gradient Gₓ along the X-axis causes the nuclei to precess at different frequencies depending on their position along the X-axis, that is, Gₓ spatially encodes the precessing nuclei by frequency, a process referred to as frequency encoding. A Y-axis gradient G_{y} is incremented through a series of values and encodes a y position in the Cartesian coordinate system into a rate of change of the phase of the precessing nuclei as a function of amplitude of the gradient G_{y}, a process referred to as phase encoding.

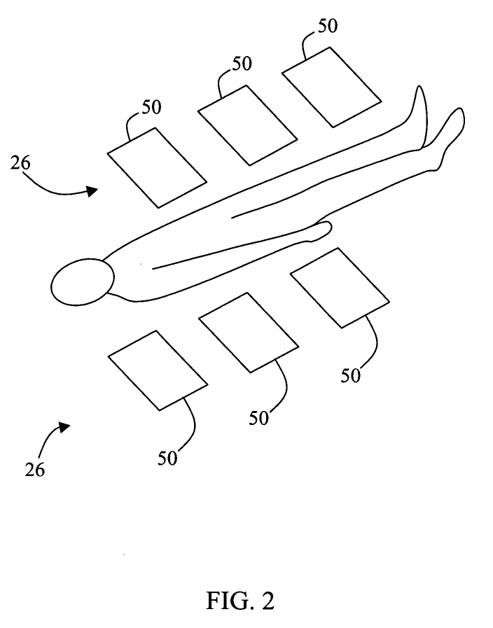

FIG. 2 illustrates an embodiment of coil arrays 26. Coil arrays 26 include one or more coils 50 arranged to detect the MR signals from subject 36. An image reconstructed with signals from an nth coil, such as coil 50, in coil array 26 is given by
Sₙ(x)=bₙ(x)M(x)+εₙ(x) (1)
where M(x) represents a magnetization of tissues of subject 36, bₙ(x) represents a coil sensitivity profile of the nth coil and an(x) denotes noise within the image.

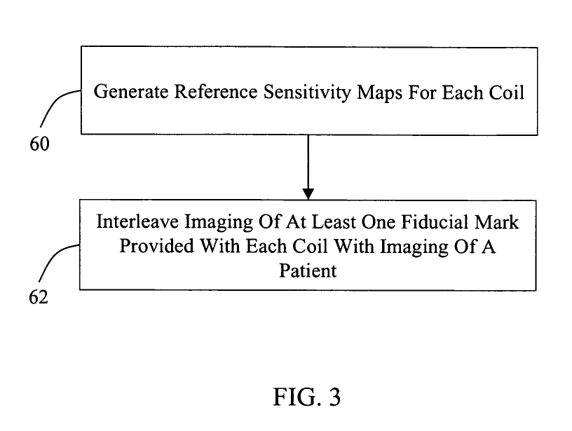

FIG. 3 is a flowchart of a method for calibrating coil sensitivity profiles that is implemented by using MRI system 10. The method includes generating 60 reference sensitivity maps or profiles one for each coil 50. In one embodiment, the reference sensitivity maps are generated by imaging a phantom placed between coil arrays 26. The image reconstructed may include an image of a fiducial mark, described below, embedded within or placed on a surface of the nth coil. If a phantom with uniform properties is placed between coil arrays 26, Sₙ(x), which is the image from the nth coil, can be used as an estimate of the reference sensitivity map. Alternatively, if a phantom with non-uniform properties is used, an image using a transmit-and-receive uniform volume coil having bₙ(x), across subject 36, substantially equal to a constant, is additionally acquired to map M(x) and Sₙ(x)/M(x) provides an estimate of the reference sensitivity map. It is noted that because the coil sensitivity profiles tend to vary slowly across a space, a spatial resolution requirement of images of the phantom used for estimating the reference sensitivity maps can be substantially lower than that of images of a patient used to diagnose the patient.

In an alternative embodiment, the reference sensitivity maps are obtained by applying Biot-Savart's law or by solving Maxwell's equations. For example, by using Biot-Savart's law, the reference sensitivity map of the nth coil can be estimated as

$${b}_{n} ( x ) = \frac{μ}{4 π} {∮}_{{C}_{n}^{′}} \frac{{ds}^{′} \times ( x - {x}^{′} )}{{\vert x - {x}^{′} \vert}^{3}} \qquad ( 2 )$$

where the line integral over a current in the nth coil is based on a filament approximation of the nth coil, where μ is a permeability constant, ds′ is an element of length along the nth coil, x-x′ is a distance in a specific direction from the element ds′ to a point at which a magnetic field is generated by a current flowing the nth coil, and “x” represents a vector product.

The method further includes interleaving 62 imaging of at least one fiducial mark embedded within each coil 50 in coil array 26 with imaging of a patient to determine positions or orientations of each coil 50 in addition to capturing changes in a coil load, referred to as coil loading changes. Coil load is an effective resistance seen by each coil 50. Coil load is dependent upon subject 36 and affects the amplitude of MR signals received by coils 50. An example of a fiducial mark is a signal generating small device. A more specific example of a fiducial mark is a small capsule filled with water.

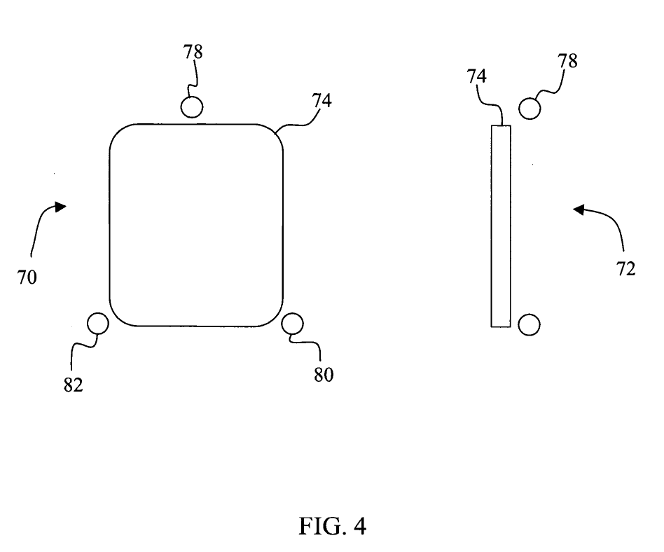

In one embodiment, images of the fiducial marks are generated by image reconstructor 22 to determine positions of coils 50 and coil loading changes. In the embodiment, a number of the fiducial marks placed on each coil 50 depends on whether coils 50 are attached to a solid former (not shown), such as a rigid or a semi-rigid bar. If coils 50 are not attached to the solid former, coils 50 are independently positioned with respect to each other and at least three fiducial marks are used with each coil 50. On the other hand, if coils 50 are affixed to the solid former, one or two fiducial marks per coil 50 are used. In the embodiment, as an example, 1-dimensional (1D) projection images of at least one fiducial mark on each coil 50 are generated by image reconstructor 22. The 1D projection images are generated by projecting signals from the fiducial marks onto a line. In the example, the fiducial marks are placed in a separate half space than a space in which the patient is placed. Such a placement in the separate half space is shown in a front view 70 and a side view 72 of a surface 74 of coil 50 in FIG. 4, where fiducial marks 78, 80, and 82 are placed on a side on surface 74 of coil 50, where the side is opposite to a side facing the patient. Such a placement facilitates isolation of signals generated from fiducial marks 78, 80, and 82 from signals generated from nuclei of the patient. The isolation is created by applying a magnetic field gradient that is orthogonal or alternatively, substantially orthogonal, to surface 74 of coil 50. In one embodiment, the step 60 is executed once before step 62.

The method also includes registering the reference sensitivity maps based on actual positions of coils 50 determined intermittently while imaging the patient and includes scaling the reference sensitivity maps based on coil loading changes also determined intermittently while imaging the patient. The registering and the scaling are performed to derive actual sensitivity profiles from the reference sensitivity maps. The actual positions of coils may be different from reference positions of coils 50. The reference positions are positions of coil 50 while generating the reference sensitivity maps, for instance, by imaging the phantom. The actual positions are calculated from coordinates of at least one fuducial mark provided with each coil 50. The coordinates are determined manually or automatically by locating associated peaks of signals from the fiducial marks in the 1D projection images of the fiducial marks. The actual positions are used to spatially register the reference sensitivity maps. The spatial registration is performed by rigidly rotating and/or translating the reference sensitivity maps to track the changes in the actual positions.

The 1D projections images of the fiducial marks are further compared to images Sₙ(x) that are reconstructed to obtain the reference sensitivity maps. A ratio of amplitudes of signals that are generated from a fiducial mark present in the images Sₙ(x) and present in the 1D projection images is calculated. For example, a first amplitude of a first signal is generated from a fiducial mark present in the images Sₙ(x) that are reconstructed to obtain the reference sensitivity maps. In the example, a second amplitude of a second signal is generated from the fiducial mark present in the 1D projection images. In the example, the ratio is a ratio of the first and the second amplitudes. The ratio reflects coil loading changes of a coil that includes the fiducial mark. A reference sensitivity map of the coil, after the spatial registration and a multiplication with this ratio, provides an estimate of an actual sensitivity profile of the coil. The actual sensitivity maps are updated periodically or at desired times, by registering and scaling the reference sensitivity maps as described above.

Technical effects of the herein described systems and methods for calibrating coil sensitivity profiles include replacing costly conventional calibration imaging steps by projection imaging of the fiducial marks while imaging the patient, where the projection imaging provides information to derive actual sensitivity profiles based on reference sensitivity maps. The reference sensitivity maps are obtained from solving Maxwell's equations or performing calibration imaging of the phantom once. By replacing the conventional calibration imaging steps, the herein described methods reduce calibration time overhead and provide coil sensitivity profiles with improved spatial coverage and update rate.

Hence, the herein described systems and methods reduce calibration overhead and reduce costly calibration imaging steps by obtaining the reference sensitivity maps and by updating the actual sensitivity maps. As described above, the actual sensitivity maps are updated by interleaving imaging of fiducial marks and the patient, where imaging of fiducial marks provides coil positioning and coil loading that are used to derive the actual sensitivity maps from the reference sensitivity maps.

An exemplary embodiment of an MRI system is described above in detail. The MRI system components illustrated are not limited to the specific embodiments described herein, but rather, components of each MRI system may be utilized independently and separately from other components described herein. For example, the MRI system components described above may also be used in combination with other imaging systems.

While the invention has been described in terms of various specific embodiments, those skilled in the art will recognize that the invention can be practiced with modification within the spirit and scope of the claims.

1. A method for calibrating coil sensitivity profiles comprising:
generating reference sensitivity maps for each coil;
imaging a subject;
interleaving, with said imaging of the subject, imaging of at least one fiducial mark provided with each coil; and
deriving, based on the coil positioning and coil loading, actual sensitivity maps from the reference sensitivity maps.

2. A method in accordance with claim 1 further comprising:
obtaining coil positioning and coil loading from said interleaving, with said imaging of the subject, imaging of at least one fiducial mark provided with each coil.

3. A method in accordance with claim 1 wherein said generating reference sensitivity maps for each coil comprises producing reference sensitivity maps for one time by imaging a phantom using a magnetic resonance imaging system.

4. A method in accordance with claim 1 wherein said generating reference sensitivity maps for each coil comprises producing the reference sensitivity maps for one time by solving Maxwell's equations.

5. A method in accordance with claim 1 wherein said interleaving, with said imaging of the subject, imaging of at least one fiducial mark provided with each coil comprises intermittently obtaining 1-dimensional projection images of the at least one fiducial mark provided with each coil while performing said imaging of the subject.

6. A method in accordance with claim 1 further comprising embedding at least one fiducial mark within each coil before said interleaving, with said imaging of the subject, imaging of at least one fiducial mark provided with each coil.

7. A method in accordance with claim 1 further comprising placing the at least one fiducial mark on each coil, wherein a number of the at least one fiducial mark depends on whether each coil is attached to a solid former.

8. A method in accordance with claim 1 further comprising spatially registering the reference sensitivity maps based on changes in position of each coil determined from said interleaving, with said imaging of the subject, imaging of at least one fiducial mark provided with each coil.

9. A method in accordance with claim 1 further comprising scaling the reference sensitivity maps based on changes in the coil loading determined from said interleaving, with said imaging of the subject, imaging of at least one fiducial mark provided with each coil.

10. A method in accordance with claim 1 further comprising applying a magnetic field gradient substantially orthogonal to a surface of each coil to perform said interleaving, with said imaging of the subject, imaging of at least one fiducial mark provided with each coil.

11. A method in accordance with claim 1 further comprising performing one of:
imaging a phantom to generate the reference sensitivity maps; and
applying Biot-Savart's law to generate the reference sensitivity maps; and
solving Maxwell's equations to generate the reference sensitivity maps.

12. A magnetic resonance imaging system comprising:
a coil array configured to receive a plurality of signals to generate magnetic resonance images, wherein said coil array is configured to obtain partial gradient phase encoding signals from a subject, said coil array is configured to intermittently receive signals from at least one fiducial mark provided with each coil of said coil array, and said coil array is configured to intermittently receive signals while obtaining the partial gradient phase encoding signals; and
an image reconstructor configured to update sensitivity maps by using the intermittently received signals and reference sensitivity maps, wherein said image reconstructor is further configured to construct magnetic resonance images based on the updated sensitivity maps and the partial gradient phase encoding signals.

13. A magnetic resonance imaging system in accordance with claim 12 further comprising a controller configured to perform one of solving Maxwell's equation and applying Biot-Savart's law to generate the reference sensitivity maps.

14. A magnetic resonance imaging system in accordance with claim 12 wherein the plurality of signals used to generate the reference sensitivity maps are signals from a phantom.

15. A magnetic resonance imaging system in accordance with claim 12 further comprising:
a magnetic field control;
a gradient field control;
a transmitter;
at least one receiver; and
a controller operationally coupled to said magnetic field control, said gradient field control, said transmitter, and said receiver, wherein said controller is configured to instruct at least one of said magnetic field control, said gradient field control, said transmitter, and said receiver to apply a pulse sequence to generate for one time the reference sensitivity maps.

16. A magnetic resonance imaging system in accordance with claim 12 wherein the reference sensitivity maps are generated before obtaining the partial gradient phase encoding signals and before intermittently receiving signals reflected from the at least one fiducial mark provided with each coil of said coil array.

17. A magnetic resonance imaging system in accordance with claim 12 wherein said image reconstructor reconstructs a 1-dimensional projection image of the at least one fiducial mark from the intermittently received signals.

18. A magnetic resonance imaging system in accordance with claim 12 wherein a number of the at least one fiducial mark provided with each coil of said coil array depends on whether each coil of said coil array is attached to a solid former.

19. A magnetic resonance imaging system in accordance with claim 12 further comprising a controller configured to spatially register the reference sensitivity maps based on changes in position of each coil determined from the at least one image reconstructed from the intermittently received signals.

20. A magnetic resonance imaging system in accordance with claim 12 further comprising a controller configured to scale the reference sensitivity maps based on changes in loading of each coil determined from at least one image reconstructed from the intermittently received signals.

21. A magnetic resonance imaging system in accordance with claim 12 further comprising a controller configured to instruct a gradient field control to energize a gradient coil, wherein said gradient coil is energized to generate a magnetic field gradient substantially perpendicular to a surface of a coil of said coil array.

22. A magnetic resonance imaging system comprising:
a coil array configured to receive a plurality of signals; and
a controller configured to generate sensitivity maps from the plurality of signals, wherein said coil array is further configured to collect partial gradient phase encoding signals from a subject, said coil array is configured to intermittently receive signals from at least one fiducial mark provided with each coil of said coil array, and said coil array is configured to intermittently receive while obtaining the partial gradient phase encoding signals.

23. A magnetic resonance imaging system in accordance with claim 22 further comprising an image reconstructor configured to update sensitivity profiles from the intermittently received signals.

24. A magnetic resonance imaging system in accordance with claim 22 wherein the sensitivity profiles are generated from reference sensitivity maps that are obtained before collecting the partial gradient phase encoding signals and before intermittently receiving signals from the at least one fiducial mark provided with each coil of said coil array.
